(19) United States
(12) Patent Application Publication
(10) Pub. No.: US 2026/0250380 A1
(43) Pub. Date: Aug. 27, 2026

(54) UNCONVENTIONAL NEW METHOD FOR SELECTIVE CELL-KILLING

(71) Applicants: Jianqiang Yu, San Diego, CA (US); Wenyun Zhou, San Diego, CA (US)

(72) Inventors: Jianqiang Yu, San Diego, CA (US); Wenyun Zhou, San Diego, CA (US)

(21) Appl. No.: 19/060,739

(22) Filed: Feb. 23, 2025

(51) Int. Cl. C07K 16/28; A61P 35/00

(52) U.S. Cl. CPC C07K 16/2803; A61P 35/00; C07K 2317/22; C07K 2317/524; C07K 2317/526; C07K 2317/622; C07K 2319/09

(57) ABSTRACT

The present invention provides an unconventional new method for selective cell-killing and/or selective cell growth inhibition, which is through continuously accumulating an external non-toxic OLS fusion antibody in an essential organelle of the target cells at the aim of clogging or over-occupying that organelle. The OLS fusion antibody comprises a cell binding moiety and an organelle localization sequence (OLS), not containing any cytotoxic payload. The cell binding moiety selectively binds to a cell surface antigen of the target cells. The OLS fragment directs OLS fusion antibody into an organelle of the target cells. The present invention provides a first-ever evidence to a new concept that continuously accumulating an external non-toxic OLS fusion antibody in an essential organelle can selectively kill the target cells and/or selectively inhibit the growth of target cells which express a specific cell surface antigen recognized by this OLS fusion antibody.

### FIELD OF THE INVENTION

The present invention relates to an unconventional new method for selectively killing cells which express a specific cell surface antigen.

### BACKGROUND OF THE INVENTION

Cancer is the second leading cause of death worldwide (Lancet 2016; Oct. 8; 388:1459-14544). There are at least four obstacles to current cancer therapy. Unintentionally targeting normal cells (which often causes severe side effects, sometimes life threatening) is the first obstacle to current cancer therapy. Antibodies (Ab), antibody drug conjugates (ADC), bi-specific T cell engagers (BiTE), chimeric antigen receptor T cells (CART) and tyrosine kinase inhibitors (TKI) are commonly used target drugs for treating cancer. Besides targeting cancer cells, these drugs also target normal cells because in many cases both cancer cells and normal cells express the same target antigen. Frequently, the expression difference between cancer cells and normal cells is that cancer cells have a high level of target antigen expression, while normal cells have a low level of target antigen expression (Leukemia volume 34, pages 985-1005 (2020); Mol Biol Int. 2014; 852748.). Current target drugs for cancer therapy, such as Ab, ADC, BiTE, CART or TKI based drugs, cannot completely differentiate the cells expressing a low level of target antigen (normal cells) from the cells expressing a high level of target antigen (cancer cells).

Redundancy of cell signal pathways (which often causes drug resistance in a short period of time) is the second obstacle to current cancer therapy. The cell has evolved for at least 3.8 billion years (The Cell: A Molecular Approach. 2nd edition. Cooper G M. Sunderland (MA): Sinauer Associates; 2000). For the sake of survival, the cell does not rely on one signal pathway and has evolved to give rise to redundant signal elements that can compensate for one another. Redundant signal elements create new and unexpected crosstalk signaling pathways that give a cell the ability to overcome the treatment effect exerted by a drug which only targets one particular signal pathway (Cancer Res. 2015 Mar. 1; 75(5): 808-812). Redundancy of cell signal pathways is one of the reasons that majority of cancer patients who achieve an initial response to trastuzumab (a cancer drug targeting HER2 signal pathway) develop resistance within one year (N Engl J Med. 2001; 344:783-92; N Engl J Med. 2012; 366:109-19).

A dysfunctional immune system (which is one of the reasons that many cancer patients are not the qualified candidates for target cancer treatments) is the third obstacle to current cancer therapy. Cancer is closely associated with a dysfunctional or weakened immune system (Nature Reviews Cancer volume 21, pages 345-359 (2021)). Current commonly used target drugs, such as Ab/BiTE/CART based drugs, depend on a functional immune system to kill cancer cells. Ab/BiTE/CART based drugs will not be fully effective if cancer patients have a dysfunctional immune system.

A suppressive tumor microenvironment (which often causes current cancer treatments ineffective) is the fourth obstacle to current cancer therapy. The tumor microenvironment includes diverse immune cell types, cancer associated fibroblasts, endothelial cells, and various additional tissue-resident cell types (Cancer Cell. 2023 Mar, 13; 41(3): 374-403). The clinical successes in cancer immunotherapy have been both astounding and unsatisfactory. Countless patients have good clinical response with cancer immunotherapy. However, many more patients have experienced minimal or no clinical benefit when given the same treatment. Accumulating evidences show that a suppressive tumor microenvironment impedes the treatment effect of current target cancer drugs (Nat Med 24, 541-550 (2018); Jin, et al Signal Transduct Target Ther. 2020 Aug. 25; 5(1):166).

Therefore, there is an unmet medical need for a new cancer treatment which is capable of killing the cells expressing a high level of target antigen (cancer cells) while saving the cells expressing a low level of target antigen (normal cells), which has less or least drug resistance caused by the redundancy of cell signal pathways, which is independent of patient's immune system (B cells, T cells, NK cells, antigen present cells, or the like), and which is less or least influenced by a suppressive tumor microenvironment.

The present invention provides an unconventional new cell-killing method which has the capability to meet the above-mentioned unmet medical need. This new cell-killing method provided in the present invention inherently has the capability to evade the aforesaid obstacles in current cancer therapy as explained in the following six paragraphs (A, B, C, D, E, and F paragraphs).

(A) It has been reported that too many ions leaking into a cell can lead to cell death (a cell death phenomenon called oncosis) (Am J Pathol. 1995Jan; 146(1):3-15). Oncosis is characterized by the influx of excessive ions in a cell. The influx of excessive ions causes the cell to swell to an abnormally large size, which ultimately leads to cell death.

(B) Based on the oncosis phenomenon, the inventors of the present invention hypothesis that if excessive external non-toxic antibodies are selectively brought into an essential organelle of the target cells (such as the nucleolus organelle, which has a highly organized structure and is vital to cell survival), they will clog or over-occupy that organelle, subsequently influence the vital cellular activities occurring in that organelle, and ultimately lead to cell death. In the present invention, an OLS fusion antibody (organelle localization sequence fusion antibody) is devised as the practical example of external non-toxic antibody to experimentally test the aforesaid hypothesis. Unlike ADC drug which depends on a cytotoxic payload (such as tubulin inhibitor) to kill the target cells, the OLS fusion antibody devised herein does not comprise any cytotoxic payload for exerting its cell-killing function. The OLS fusion antibody devised herein comprises: (i) a cell binding moiety, which is a biparatopic bi-specific single chain antibody; and (ii) an organelle localization sequence (OLS), which contains a non-classical nuclear localization sequence (NLS) and a non-classical nucleolar localization sequence (NoLS). In this exemplified OLS fusion antibody, (i) the cell binding moiety is designed to specifically bind a cell surface antigen at two non-overlap epitopes. This specific binding is designed to cross-link a cell surface antigen through two non-overlap epitopes, and subsequently to cause OLS fusion antibody to be internalized into the target cells; (ii) the NLS is designed to direct OLS fusion antibody into nucleus after the cellular internalization of OLS fusion antibody; (iii) the NoLS is designed to further direct OLS fusion antibody into nucleolus after OLS fusion antibody entering nucleus. The nucleolus has a highly organized structure and is vital for cell division, growth and survival (The Functional Nucleus. 2016 Apr. 23:29-49; Biochimica et Biophysica Acta 1842 (2014) 798-801). Thus, when more and more external non-toxic OLS fusion antibodies selectively and continuously accumulate in the nucleolus of target cells through retrograde protein trafficking route, the vital cellular activities occurring in nucleolus will be influenced, which will subsequently and ultimately lead to cell death. Unlike many other cancer drugs which kill cancer cells through targeting a particular cell signal pathway (such as HER2 signal pathway), the OLS fusion antibody devised herein kills cancer cells through selective and continuous accumulation in an essential organelle of the target cells at the aim of clogging or over-occupying that organelle.

(C) There is two categories of NLS and NoLS: classical and non-classical. Both classical NLS and classical NoLS comprise a cluster of positively charged amino acids (Arginine, Lysine and Histidine) (Lu, J. etal Cell Commun Signal 19, 60(2021); Nucleus. 2015; 6(4)314-325). It has been extensively reported that the peptide comprising a cluster of positively charged amino acids might work as a cell penetrating peptide (CPP), and that a cell penetrating peptide (CPP) has the capability to non-selectively traverse across cell's plasma membrane (Chemistry & Biology, vol. 8, 943-948 August 2002; J Biol Chem. 2018 Sept. 28; 293(39): 15221-15232). Although highly efficient in mediating uptake of different molecules into a cell, the use of CPP turns out to be unsuccessful mainly because of a lack of cell specificity (Mol Biotechnol. 65, 1387-1402). Therefore, in order to avoid that the OLS portion (which contains a NLS and a NoLS) of OLS fusion antibody devised herein might have the non-specific cell penetrating function (CPP function), and that the OLS fusion antibody devised herein might non-selectively traverse across cell's plasma membrane, a HnRNPA1-derived non-classical NLS and a NPM1-derived non-classical NoLS are chosen for the construction of OLS fusion antibody, because HnRNPA1's NLS (NP-002127.1, aa 268-aa 305) and NPM1's NoLS (NP-002511.1, aa 258-aa 294) do not contain a cluster of positively charged amino acids (Arginine, Lysine and Histidine). If OLS fusion antibody's OLS portion does not have a cell penetrating function (CPP function), the specific binding between OLS fusion antibody's cell binding moiety portion and target cell's specific surface antigen will be the route for OLS fusion antibody to pass through cell's plasma membrane and enter into the target cells, which will ensure the selectivity of cell-killing exerted by this OLS fusion antibody.

(D) The OLS fusion antibody (organelle localization sequence fusion antibody) devised herein comprises an antibody portion (cell binding moiety portion) and an OLS fragment. The CPP fusion antibody (cell penetrating peptide fusion antibody, which has been published in many literatures) comprises an antibody portion and a CPP fragment (Sci Rep. 2019 Dec. 10; 9(1):18688; Theranostics. 2018 Jan. 1; 8(2):549-562). Both OLS fragment and CPP fragment are short peptides. From the perspective of structure, OLS fusion antibody and CPP fusion antibody are similar to each other or the same. However, these two fusion antibodies work on different mechanisms and are totally two different things. The OLS fusion antibody devised herein crosses cell's plasma membrane through the specific binding between OLS fusion antibody's cell binding moiety portion and target cell's specific surface antigen. OLS fusion antibody's OLS fragment does not perform the activity of penetrating cell plasma membrane and does not play a role in OLS fusion antibody entering into the target cells from the outside of cell plasma membrane, which does not abolish or reduce the selectivity of OLS fusion antibody. In contrast, CPP fusion antibody's CPP fragment performs the activity of penetrating cell plasma membrane and plays a role in CPP fusion antibody entering into the target cells from the outside of cell plasma membrane, which abolishes or reduces the selectivity of CPP fusion antibody. Therefore, the OLS fusion antibody devised herein is different from the CPP fusion antibody which has appeared in many research literatures for a long time.

(E) This new method of cell-killing executed by the OLS fusion antibody devised herein is an unconventional method. The conventional cell-killing methods used in current cancer treatments are: (i) antibodies (Ab) kill cancer cells through antibody dependent cell-mediated cytotoxicity, complement-dependent cytotoxicity and antibody dependent phagocytosis (Nature reviews, Cancer 12, 278-287); (ii) antibody drug conjugates (ADC) kill cancer cells through a cytotoxic payload (such as tubulin inhibitor) (Front Pharmacol 2023; 14:1274088); (iii) BiTE and CART kill cancer cells through activating specific T cells (J Hematol Oncol May 3; 14, 75(2021); Blood Cancer J. 11, 69(2021)); (iv) checkpoint inhibitors (such as PD-1 antibody) kill cancer cells through blocking a protein (such as PD-1) that regulates the function of immune system (J Clin Med. 2023 Jun. 27; 12(13):4301); (v) chemotherapies or radiotherapies kill cancer cells through the induction of DNA damage by chemical or radioactive reagents (Front. Oncol. 12:960317; MedComm (2020) Sep; 2(3); 315-340). In comparison, this new method of cell-killing provided herein is through selectively and continuously accumulating an external non-toxic OLS fusion antibody in an essential organelle of the target cells at the aim of clogging or over-occupying that organelle (herein, the essential organelle likes a fine instrument and the external non-toxic OLS fusion antibody likes a debris, the fine instrument will be broken if too many debris are brought into its delicate internal). Therefore, this new cell-killing method provided herein is totally different from the above-mentioned conventional cell-killing methods. This new cell-killing method provided in the present invention is an unconventional cell-killing method.

(F) This unconventional new cell-killing method executed by the OLS fusion antibody devised herein is capable of evading the aforesaid obstacles in current cancer therapy and meeting the above-mentioned unmet medical need: (i) this unconventional new cell-killing method has the capability to differentiate the cells expressing a high level of cell surface antigen (cancer cells) from the cells expressing a low level of cell surface antigen (normal cells). In this new cell-killing method, the specific binding between a cell binding moiety and a cell surface antigen determines the cellular internalization of OLS fusion antibody and subsequently the effect of cell-killing. The cells expressing a high level of cell surface antigen have the capacity to transport enough external non-toxic OLS fusion antibodies into the essential organelle, which will ultimately lead to cell death. The cells expressing a low level of cell surface antigen do not have the capacity to transport enough external non-toxic OLS fusion antibodies into the essential organelle, which will not lead to cell death; (ii) this unconventional new cell-killing method has the capability to evade drug resistance which is caused by the redundancy of cell signaling pathways. The process of this new cell-killing method is: an external non-toxic OLS fusion antibody binds to a specific cell surface antigen, internalized into the target cells, continuously accumulates in an essential organelle of the target cells, and ultimately leads to cell death. As long as the specific antigen exists on the surface of target cells in enough quantity and can be bound by OLS fusion antibody, the OLS fusion antibody will be able to exert its cell-killing function after adequately accumulating in an essential organelle of the target cells, no matter how the target cells evolve to generate new bypassing cell signaling pathways; (iii) this unconventional new cell-killing method is independent of patient's immune system (T cells, B cells, NK cells, antigen present cells, or the like), because this new cell-killing method depends on continuously accumulating an external non-toxic OLS fusion antibody in an essential organelle of the target cells, this new cell-killing method does not rely on any type of immune cells; (iv) this unconventional new cell-killing method is less or least influenced by a suppressive tumor microenvironment. Current cancer treatments are significantly influenced by a suppressive tumor microenvironment (Cancer Lett 2017 Feb. 28:387:61-68). In contrast, for this unconventional new cell-killing method, as long as the target antigen exists on the cell surface in enough quantity and can be bound by OLS fusion antibody, the OLS fusion antibody will be able to exert its cell-killing function after adequately accumulating in an essential organelle of the target cells, no matter how tumor microenvironments behave or change.

Besides being applicable for killing cancer cells, this unconventional new cell-killing method executed by the OLS fusion antibody devised herein may also be applicable for: (i) killing target cells infected by organisms (such as viruses, bacteria, fungi, or parasites), as long as the infected cells can express an antigen, which is derived from the infectious organisms and recognized by the OLS fusion antibody, on the cell surface; (ii) killing target cells associated with or related to a disease (such as an immune disease), as long as the target cells can express a specific antigen, which is recognized by the OLS fusion antibody, on the cell surface.

### BRIEF DESCRIPTION OF THE DRAWINGS

FIG. 1 illustrated the structure of OLS fusion antibodies. In FIG. 1, N was referred to the amino terminus of a cell binding moiety; C was referred to the carboxy terminus of a cell binding moiety; OLS represented the organelle localization sequence.

FIG. 2 illustrated the structure of anti-Ror1 single chain antibodies (AR1, AR2, AR bi-scfv OLS fusion antibody, AR bi-scfv antibody, and AR bi-scfv OLS GFP fusion antibody). As shown in FIG. 2, (i) AR1 contained: 4A5VL (4A5 light chain variable domain), amino acids linker, 4A5VH (4A5 heavy chain variable domain), mCH2CH3 (mouse IgG heavy chain constant region 2 and region 3); (ii) AR2 contained: 99961VL (99961 light chain variable domain), amino acids linker, 99961VH (99961 heavy chain variable domain), mCH2CH3; (iii) AR bi-scfv OLS fusion antibody contained: 4A5VL, amino acids linker, 99961VL, amino acids linker, 99961VH, amino acids linker, 4A5VH, NLS, hCH2CH3 (human IgG heavy chain constant region 2 and region 3), NoLS; (iv) AR bi-scfv antibody contained: 4A5VL, amino acids linker, 99961VL, amino acids linker, 99961VH, amino acids linker, 4A5VH, hCH2CH3; (v) AR bi-scfv OLS GFP fusion antibody contained: 4A5VL, amino acids linker, 99961VL, amino acids linker, 99961VH, amino acids linker, 4A5VH, NLS, hCH2CH3, NoLS, GFP. In FIGS. 2, 4A5 and 99961 were the names of two full anti-Ror1 antibodies; NLS represented the nuclear localization sequence; NoLS represented the nucleolar localization sequence; GFP represented the green fluorescent protein; AR was referred to anti-Ror1; scfv represented single chain fragment variable.

In FIG. 3, (i) line 1 showed the result of P815 mock cells stained with PE Mouse Anti-Human Ror1 mAb; (ii) line 2 showed the result of P815Ror1 cells (clone 4C1) stained with PE Mouse Anti-Human Ror1 mAb; (iii) line 3 showed the result of P815Ror1 cells (clone 6B2) stained with PE Mouse Anti-Human Ror1 mAb.

In FIG. 4, (i) line 1 (dash line) showed the result of P815AR bi-scfv cells stained with FITC-Labeled negative control protein; (ii) line 2 (solid line) showed the result of P815AR bi-scfv cells stained with FITC-Labeled human Ror1 protein.

In FIG. 5, (i) dash line showed the result of Jeko-1 cells stained with secondary antibody only; (ii) solid line showed the result of Jeko-1 cells first stained with AR1 or AR2, then stained with secondary antibody.

FIG. 6 showed: (i) in experiment 1, cells were stained with AR bi-scfv OLS fusion antibody; (ii) in experiment 2, cells were first stained with AR1, then stained with AR bi-scfv OLS fusion antibody; (iii) in experiment 3, cells were first stained with AR2, then stained with AR bi-scfv OLS fusion antibody; (iv) in experiment 4, cells were first stained with both AR1 and AR2, then stained with AR bi-scfv OLS fusion antibody. Dash line showed the result of P815 mock cells (Ror1 negative cells) stained with corresponding antibodies. Solid line showed the result of P815Ror1 cells (Ror1 positive cells) stained with corresponding antibodies.

FIG. 7 (Cytotoxicity Assay 1) showed: (i) in the left side picture, P815 mock cells (Ror1negative cells) were treated with AR bi-scfv OLS fusion antibody for 12 days, then stained by Trypan blue; (ii) in the middle picture, P815Ror1 cells (clone 4C1) were treated with AR bi-scfv OLS fusion antibody for 12 days, then stained by Trypan blue; (iii) in the right side picture, P815Ror1 cells (clone 6B2) were treated with AR bi-scfv OLS fusion antibody for 12 days, then stained by Trypan blue.

FIG. 8 (Cytotoxicity Assay 2) showed: (i) in the left side picture, P815Ror1 cells were treated with AR bi-scfv antibody (an antibody without OLS fragment) for 12 days, and then stained by Trypan blue; (ii) in the right side picture, P815Ror1 cells were treated with AR bi-scfv OLS fusion antibody (an antibody with OLS fragment) for 12 days, and then stained by Trypan blue.

FIG. 9 showed that AR bi-scfv OLS GFP fusion antibody was observed both on the cell surface and in the nucleolus after treating P815Ror1 cells for 12 days.

### SUMMARY OF THE INVENTION

The unconventional new method of cell-killing disclosed in the present invention is a method of selectively transferring an external non-toxic OLS fusion antibody (organelle localization sequence fusion antibody) from the outside of target cells (that is, the outside of plasma membrane) into an essential organelle at the aim of clogging or over-occupying that organelle. The said OLS fusion antibody comprises a cell binding moiety and an organelle localization sequence (OLS).

### 1. Cell Binding Moiety

In one embodiment, the cell binding moiety of OLS fusion antibody may be, but not limited to, an antibody, an antigen-binding fragment, a protein scaffold, a cytokine, or a ligand for receptor.

In one embodiment, the cell binding moiety of OLS fusion antibody may be a mono-specific cell binding moiety, preferably a mono-specific antibody or mono-specific antigen-binding fragment, wherein the said a mono-specific cell binding moiety specifically binds to a cell surface antigen.

In one embodiment, the cell binding moiety of OLS fusion antibody may be a bi-specific cell binding moiety, preferably a bi-specific antibody or bi-specific antigen-binding fragment, wherein two parts of the said a bi-specific cell binding moiety specifically bind to the same cell surface antigen but at different epitopes, or wherein two parts of the said a bi-specific cell binding moiety specifically bind to two different cell surface antigens.

In one embodiment, the cell binding moiety of OLS fusion antibody may be a bi-specific cell binding moiety, preferably a bi-specific antibody or bi-specific antigen-binding fragment, wherein the first part of said a bi-specific cell binding moiety specifically binds to a cell surface antigen, while the second part of said a bi-specific cell binding moiety specifically binds to an intracellular antigen. After internalization, the OLS fusion antibody is able to specifically bind an intracellular antigen through the second part of said a bi-specific cell binding moiety.

In one embodiment, the cell binding moiety of OLS fusion antibody may be a tri-specific cell binding moiety, preferably a tri-specific antibody or tri-specific antigen-binding fragment, wherein three parts of the said a tri-specific cell binding moiety specifically bind to the same cell surface antigen but at different epitopes, or wherein three parts of the said a tri-specific cell binding moiety specifically bind to three different cell surface antigens.

In one embodiment, the cell binding moiety of OLS fusion antibody may be a tri-specific cell binding moiety, preferably a tri-specific antibody or tri-specific antigen-binding fragment, wherein one or two parts of the said a tri-specific cell binding moiety specifically bind to a cell surface antigen or antigens, while the other one or two parts of said a tri-specific cell binding moiety specifically bind to an intracellular antigen or antigens. After internalization, the OLS fusion antibody is able to specifically bind an intracellular antigen or antigens through the other one or two parts of said a tri-specific cell binding moiety.

In one embodiment, the cell binding moiety of OLS fusion antibody may be a multi-specific cell binding moiety, preferably a multi-specific antibody or multi-specific antigen-binding fragment, wherein multiple parts of the said a multi-specific cell binding moiety specifically bind to the same cell surface antigen but at different epitopes, or wherein multiple parts of the said a multi-specific cell binding moiety specifically bind to multiple different cell surface antigens.

In one embodiment, the cell binding moiety of OLS fusion antibody may be a multi-specific cell binding moiety, preferably a multi-specific antibody or multi-specific antigen-binding fragment, wherein one part or some parts of the said a multi-specific cell binding moiety specifically bind to a cell surface antigen or antigens, while other part or parts of the said a multi-specific cell binding moiety specifically bind to an intracellular antigen or antigens. After internalization, the OLS fusion antibody is able to specifically bind an intracellular antigen or antigens through the other part or parts of said a multi-specific cell binding moiety.

In one embodiment, the effect of cell-killing may be achieved by using the cell binding moiety of OLS fusion antibody to target one cell surface antigen. In one embodiment, the effect of cell-killing may be achieved by using the cell binding moiety of OLS fusion antibody to target two or more antigens, wherein the said antigens are all presented on cell surface, or wherein some of said antigens are presented on cell surface, while others exist in cell's internal. In one embodiment, the effect of cell-killing may be achieved by increasing the molecular size of a cell binding moiety because the bigger cell binding moiety may clog or over-occupy an essential organelle better than the smaller cell binding moiety.

In one embodiment, the cell binding moiety of OLS fusion antibody may be one isotype of an antibody, including IgA, IgG, IgM, IgD, or IgE.

In one embodiment, the cell binding moiety of OLS fusion antibody may be an antibody, wherein said antibody may be a, without limitation, human, humanized, non-human, and/or chimeric antibody, and wherein said antibody may be based on an antibody from a mammal, bird, fish, amphibian, or reptile.

In one embodiment, the cell binding moiety of OLS fusion antibody may be an antigen-binding fragment, wherein said antigen-binding fragment may be a, without limitation, human, humanized, non-human, and/or chimeric fragment, and wherein said antigen-binding fragment may be based on an antibody or a protein from a mammal, bird, fish, amphibian, or reptile.

In one embodiment, the cell binding moiety of OLS fusion antibody may be a protein scaffold, wherein said protein scaffold may be a, without limitation, human, humanized, non-human, and/or chimeric scaffold, and wherein said protein scaffold may be based on a protein from a mammal, bird, fish, amphibian, or reptile.

In one embodiment, it is particularly preferred that through its cell binding moiety, the OLS fusion antibody can tightly bind to a target antigen with super high affinity (preferably in low femto-molar affinity). In one embodiment, it is particularly preferred that the OLS fusion antibody can tightly bind to a target antigen through its cell binding moiety and drag that target antigen into an essential organelle of the target cells at the aim of clogging or over-occupying that organelle together.

In one embodiment, it is mandatory that the specific binding between a cell binding moiety and a cell surface antigen can lead to the cellular internalization of OLS fusion antibody. In one embodiment, it is preferred to select or screen a suitable cell binding moiety, which is an internalization-able cell binding moiety after binding to a cell surface antigen, for constructing the OLS fusion antibody.

In one embodiment, it is preferred that the OLS fusion antibody itself cannot pass through the plasma membrane of target cells without the specific binding between its cell binding moiety and a cell surface antigen. In one embodiment, it is preferred that the OLS fusion antibody can pass through the plasma membrane of target cells only after the specific binding between its cell binding moiety and a cell surface antigen.

In one embodiment, the cell binding moiety of OLS fusion antibody specifically binds to a cell surface protein, such as a cell surface receptor. In one embodiment, the cell binding moiety specifically binds to a cancer associated antigen or cancer antigen which is presented on the surface of target cells. In one embodiment, the cell binding moiety specifically binds to an autoimmune disease associated antigen or autoimmune disease antigen which is presented on the surface of target cells. In one embodiment, the cell binding moiety specifically binds to an infectious disease associated antigen or infectious disease antigen which is presented on the surface of target cells.

### 2. Organelle Localization Sequence (OLS)

In one embodiment, an organelle targeted by the OLS portion of OLS fusion antibody may be, but not limited to, a nucleolus, a mitochondrion, an endoplasmic reticulum, a nucleus, a Golgi apparatus, or a lysosome. In one embodiment, it is preferred that the organelle targeted by the OLS portion of OLS fusion antibody has a highly organized structure and is vital to cell survival. In one embodiment, after the cellular internalization of OLS fusion antibody, the function of OLS portion is to direct OLS fusion antibody into an organelle of the target cells.

In one embodiment, the OLS portion of OLS fusion antibody may be a nuclear localization sequence (NLS) as long as the NLS fragment does not have a cell penetrating peptide function (CPP function, which is capable of non-selectively penetrating the plasma membrane of a cell). In one embodiment, the OLS portion may be a nucleolar localization sequence (NoLS) as long as the NoLS fragment does not have a CPP function. In one embodiment, the OLS portion may be a combination of nuclear localization sequence and nucleolar localization sequence as long as the combined sequences do not have a CPP function. In one embodiment, the OLS portion may be an endoplasmic reticulum localization sequence (ErLS) as long as the ErLS fragment does not have a CPP function. In one embodiment, the OLS portion may be a mitochondrion localization sequence (MLS) as long as the MLS fragment does not have a CPP function. In one embodiment, the OLS portion may be a lysosome localization sequence (LLS) as long as the LLS fragment does not have a CPP function. In one embodiment, the OLS portion may be a Golgi apparatus localization sequence (GaLS) as long as the GaLS fragment does not have a CPP function.

In one embodiment, the OLS portion of OLS fusion antibody may be comprised in the N terminus of a cell binding moiety, may be comprised in the C terminus of a cell binding moiety, may be comprised in both N terminus and C terminus of a cell binding moiety, may be comprised in the middle of a cell binding moiety, or may be comprised in a random site of the cell binding moiety.

In one embodiment, one, two, three, or more organelle localization sequences (OLSs) may be used in constructing one OLS fusion antibody, wherein said organelle localization sequences (OLSs) target the same organelle, or wherein said organelle localization sequences (OLSs) target different organelles.

In one embodiment, the OLS portion of OLS fusion antibody may be genetically added to the cell binding moiety portion, or chemically conjugated to the cell binding moiety portion, as long as the conjugation procedure does not affect both OLS fragment's function and cell binding moiety's function.

In one embodiment, it is mandatory that the OLS portion of OLS fusion antibody does not have a cell penetrating peptide function (CPP function). In one embodiment, one or more point mutations may be needed to delete the unwanted feature of original OLS, such as the feature of CPP. In one embodiment, through point mutation or mutations to delete OLS' original cell penetrating function, the OLS fusion antibody will enter into the target cells through the specific binding between OLS fusion antibody's cell binding moiety portion and target cell's surface antigen.

In one embodiment, the organelle localization sequence (OLS) may be covalently linked to a cell binding moiety with one linker. In one embodiment, one or more linkers may be used to connect the organelle localization sequence (OLS) to a cell binding moiety.

In one embodiment, the organelle localization sequence (OLS) may be connected to a cell binding moiety using any of a variety of known and established techniques, wherein said techniques rely upon standard chemical, biochemical, and/or molecular techniques.

### 3. A Pharmaceutical Composition Comprising OLS Fusion Antibody

In one embodiment, the present invention provides a pharmaceutical composition using the OLS fusion antibody as an active ingredient, wherein said OLS fusion antibody comprises a cell binding moiety and an organelle localization sequence (OLS), and wherein said OLS fusion antibody exerts its function through continuous accumulation in an organelle of the target cells at the aim of clogging or over-occupying that organelle.

In one embodiment, a pharmaceutical composition comprising the OLS fusion antibody may be used to treat a disease or disorder in a subject in need thereof, as long as the disease or disorder is associated with or related to a specific cell surface antigen recognized by the OLS fusion antibody and the treatment purpose is to deplete the particular cells which express that specific cell surface antigen. In one further embodiment, the said a disease or disorder may be a cancer, wherein said cancer may be, but not limited to, breast cancer, colorectal cancer, rectal cancer, lung cancer, colon cancer, thyroid cancer, oral cancer, pharyngeal cancer, laryngeal cancer, cervical cancer, brain cancer, ovarian cancer, bladder cancer, kidney cancer, liver cancer, pancreatic cancer, prostate cancer, skin cancer, tongue cancer, uterine cancer, gastric cancer, bone cancer, blood cancer, or the like. In one further embodiment, the said a disease or disorder may be an infectious disease, wherein said an infectious disease may be, but not limited to, virus infection, bacterial infection, fungus infection, or parasite infection. In a particular embodiment, the virus infection may be, but not limited to, HBV infection, HPV infection, or HIV infection. In one further embodiment, the said a diseases or disorder may be an autoimmune disease, wherein said an autoimmune disease may be, but not limited to, rheumatoid arthritis, type 1 diabetes, multiple sclerosis, or psoriasis.

In one embodiment, a pharmaceutical composition comprising the OLS fusion antibody may be used for treating cell populations in culture to diminish a subtype of cell. In mixed cultures, where one wishes to avoid interference by a subtype of cell, this may be achieved by adding a pharmaceutical composition comprising the OLS fusion antibody to remove substantially all of a subtype of cell present in the culture. In a similar manner, therapeutic treatments may be utilized in which blood is removed from a patient into an external environment (as in dialysis), treated with a pharmaceutical composition comprising the OLS fusion antibody to remove a subtype of cell, and then returned to the patient.

In one embodiment, a pharmaceutical composition comprising the OLS fusion antibody may be used for selectively killing the cells expressing a high level of cell surface antigen which is recognized by the OLS fusion antibody, while saving the cells expressing the same cell surface antigen but at a low level.

In one embodiment, in order to effectively kill the target cells, the pharmaceutical composition may comprise a combination of OLS fusion antibodies which respectively target different cell surface antigens. For example, a pharmaceutical composition comprising several OLS fusion antibodies may be administered to an individual with a disease or disorder, such as an individual suspected of developing drug resistance when treated with a pharmaceutical composition comprising only one type of OLS fusion antibody.

In one embodiment, the pharmaceutical composition may be a polynucleotide (such as a mRNA molecule) which encodes the OLS fusion antibody. In one embodiment, the pharmaceutical composition may be an expression vector which expresses the OLS fusion antibody. In one embodiment, the pharmaceutical composition may be a cell which comprises a polynucleotide encoding the OLS fusion antibody.

### 4. Production of OLS Fusion Antibody

The OLS fusion antibody may be manufactured according to the conventional publicly-known gene engineering technique. For example, DNA encoding cell binding moiety and DNA encoding organelle localization sequence (OLS) are amplified if necessary, those DNAs are bound each other by PCR amplification, the resulting DNA is inserted into a cellular expression vector and a host cell is transfected with the vector to express OLS fusion antibody whereby the OLS fusion antibody can be manufactured. As to the expression host cell, mammalian cell, yeast, animal cell, insect cell, plant cell, or bacterial cell, may be used. Among them, animal cell is preferred, and CHO cell or HEK293 cell is particularly preferred. Standard techniques may be used for recombinant DNA, tissue culture, transformation, transfection, protein expression, and protein purification. Antibody expression and purification techniques may be performed according to manufacturer's specifications or as commonly accomplished in the related art. These and related techniques and procedures may be generally performed according to conventional methods well known in the art.

### DETAILED DESCRIPTION OF THE INVENTION

Before the present method and composition are described, it will be appreciated by those skilled in the art that changes could be made to the embodiments described above without departing from the broad inventive concept. It is understood, therefore, that this invention is not limited to the particular embodiments disclosed. Unless otherwise defined, all technical and scientific terms used herein have the same meaning as commonly understood by one of ordinary skill in the art to which this invention belongs. It is also to be understood that the terminology used herein is for the purpose of describing particular embodiments only, and is not intended to be limiting. These and other aspects of the present invention will become apparent upon reference to the following detailed description and attached drawings. Examples are put forth so as to provide those of ordinary skill in the art with a complete disclosure and description of how to make and use the present invention, and are not intended to limit the scope of the present invention.

### I. Definition

Terms such as “a”, “an”, “the” and “one” are not intended to only mean a singular entity, but include the general class of which a specific example may be used for illustration unless the context clearly dictates otherwise.

Throughout this specification, unless the context requires otherwise, the word “comprise,” or variations such as “comprises” or “comprising,” will be understood to imply the inclusion of a stated element or integer or group of elements or integers but not the exclusion of any other element or integer or group of elements or integers.

The term “or” is used herein to mean, and is used interchangeably with, the term “and/or”, unless the context clearly indicates otherwise.

The terms “specific”, “specifically binds” and “binds specifically” are used herein to mean the selective binding between a cell binding moiety and a target antigen. The binding specificity of a cell binding moiety may be tested by comparing the binding to an appropriate antigen to the binding to an irrelevant antigen under a given set of conditions.

The term “cell” is referred to the smallest unit that can live on its own and that makes up all living organisms and the tissues of the body. A mammalian cell has three main parts: cell plasma membrane, nucleus, and cytoplasm.

The term “organelle” is used herein to mean a specialized subunit within a cell that has a specific function. An organelle has one or more specific jobs to perform in the cell, much like an organ does in the body. Examples of organelle include: nucleus, mitochondrion, endoplasmic reticulum, Golgi apparatus, nucleolus, and lysosome.

The term “binding moiety” is used herein to mean a molecule or a portion of a molecule which binds to a target molecule. The term “cell binding moiety” is used to mean a molecule or a portion of a molecule which binds to a target molecule expressed by a cell.

The term “antigen” is used herein to mean a molecule that is capable of stimulating a host's immune system to make cellular and/or humoral antigen-specific immune responses when this molecule is administered into the host. The term “cell surface antigen” is used to mean an antigen expressed on the surface of a cell that can be targeted with a cell binding moiety. Cell surface antigens comprise not only those encoded by the cell itself but also the products of intracellular virus, bacterium, fungus, or parasite.

The term “antibody” herein is used in the broadest sense and specifically covers natural antibody and engineered antibody, so long as they exhibit the desired biological activity. The term “antibody” also encompasses Ig molecules formed only from a heavy chain, such as those obtained from Camelids. The antibody may be of any type or class (e.g., IgG, IgE, IgM, IgD, and IgA) or subclass (e.g., IgG1, IgG2, IgG3, IgG4, IgA1 and IgA2). The antibody may be derived from any suitable species.

The term “antigen-binding fragment” is used herein to mean a polypeptide comprising a portion specifically binding to the antigen. Without limitation, the antigen-binding fragment may be a heavy chain complementary determining region (CDR), a light chain CDR, a heavy chain variable region, a light chain variable region, or a combination thereof. The antigen-binding fragment may be a Fab fragment, a Fv fragment, a dAb fragment, a F(ab′) 2 fragment, a single chain fragment, a dia-body, a bi-specific antibody, a tri-body, a tetra-body, a linear antibody, a cytokine, or a ligand for receptor. The antigen-binding fragment may be the protein scaffold, a protein structure having a structure similar to that of an antibody other than an antibody and/or having a characteristic of specifically recognizing and/or binding a specific antigen. The antigen-binding fragment may comprise any protein or portion thereof that retains the ability to specifically bind an antigen, including non-immunoglobulin.

The term “organelle localization sequence (OLS)” is used herein to mean an amino acid sequence that directs a protein for import into an organelle of the target cell. In the present invention, the term “localization sequence” is same as the term “localization signal” which is often used in scientific literatures and text books.

The term “nuclear localization sequence (NLS)” is used herein to mean an amino acid sequence that directs a protein for import into the nucleus of a cell. The term “nucleolar localization sequence (NoLS)” is used to mean an amino acid sequence that directs a protein for import into the nucleolus of a cell. The term “mitochondrion localization sequence (MLS)” is used to mean an amino acid sequence that directs a protein for import into the mitochondrion of a cell. The term “Golgi apparatus localization sequence (GaLS)” is used to mean an amino acid sequence that directs a protein for import into the Golgi apparatus of a cell. The term “endoplasmic reticulum localization sequence (ErLS)” is used to mean an amino acid sequence that directs a protein for import into the endoplasmic reticulum of a cell. The term “lysosome localization sequence (LLS)” is used to mean an amino acid sequence that directs a protein for import into the lysosome of a cell.

The term “OLS fusion antibody” is used herein to represent the organelle localization sequence (OLS) fusion antibody which comprises a cell binding moiety and an OLS fragment. Examples of OLS fragment include: nuclear localization sequence (NLS), nucleolar localization sequence (NoLS), mitochondrion localization sequence (MLS), Golgi apparatus localization sequence (GaLS), lysosome localization sequence (LLS), and endoplasmic reticulum localization sequence (ErLS). The term “non-toxic OLS fusion antibody” is used to mean an OLS fusion antibody which does not comprise any cytotoxic payload such as tubulin inhibitor, topoisomerase I inhibitor, or the like.

The term “non-classical nuclear localization sequence (NLS)” is used herein to mean an amino acid sequence that directs a protein for import into the nucleus of a cell, but does not follow the typical pattern of a classical nuclear localization sequence (NLS), which usually consists of a cluster of positively charged amino acids (Arginine, Lysine and Histidine).

The term “non-classical nucleolar localization sequence (NoLS)” is used herein to mean an amino acid sequence that directs a protein for import into the nucleolus of a cell, but does not follow the typical pattern of a classical nucleolar localization sequence (NoLS), which usually consists of a cluster of positively charged amino acids (Arginine, Lysine and Histidine).

The term “a polynucleotide” is used herein to mean a combination of nucleotide monomers which are connected to each other through covalent bonds. DNA (deoxyribonucleic acid) and RNA (ribonucleic acid) are examples of a polynucleotide.

The term “disease” is used herein to mean a medical condition that has a clear cause. The term “disorder” is used herein to mean a disruption of normal health condition, the cause of which can be unknown or subjective.

The term “cancer” is used herein to mean a disease in which abnormal cells divide without control and can invade nearby tissues through the blood and lymph systems. The term “tumor” is used to mean a swelling of a part of the body, caused by the abnormal growth of a tissue. The term “autoimmune disease” is used to mean a condition in which the body's immune system mistakes its own tissues as the foreign and attacks them. The term “infectious disease” is used to mean a condition caused by organisms such as bacteria, viruses, fungi, or parasites.

The terms “treat,” “treatment” and “therapy” as used within the context of the present invention, are meant to include therapeutic as well as prophylactic, or suppressive measures for a disease or disorder leading to any clinically desirable or beneficial effect, including but not limited to alleviation of one or more symptoms, regression, slowing or cessation of progression of the disease or disorder.

### II. Example

### 1. Construction of OLS Fusion Antibody

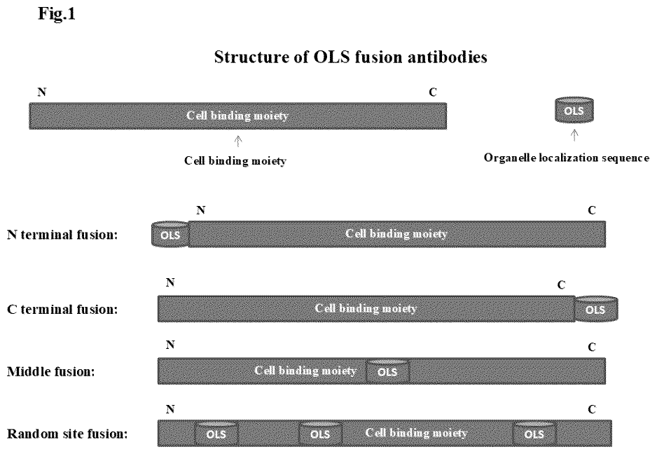

The OLS fusion antibody devised in the present invention comprised a cell binding moiety and an organelle localization sequence (OLS). As shown in FIG. 1, for constructing OLS fusion antibody, the OLS fragment may be added to: (i) the N terminus of a cell binding moiety; (ii) the C terminus of a cell binding moiety; (iii) both N and C terminus of a cell binding moiety; (iv) the middle of a cell binding moiety; (v) a random site of the cell binding moiety, as long as there was no mutual interference between OLS' function and cell binding moiety's function. In the present invention, an anti-Ror1 biparatopic bi-specific scfv OLS fusion antibody was designed and constructed as the practical example of OLS fusion antibody to test the new cell-killing method.

### 2. Production of Anti-Ror1 Mono-specific Scfv Antibodies (AR1 and AR2)

Human Ror1 protein (NP_005003, a cell surface protein) was selected herein as the target antigen to test the cell-killing effect of OLS fusion antibody, because Ror1 protein was highly expressed in a variety of cancer cells, but not expressed or expressed at a very low level in normal cells. Ror1 protein, a type I trans-membrane protein with 937 amino acids at 104 KD molecular mass, had been reported being able to inhibit apoptosis, potentiate EGFR signaling, and promote cell growth (Hematol Oncol 2011, 29:17-21; Protein & Cell, Volume 5, issue 7, July 2014, 496-502).

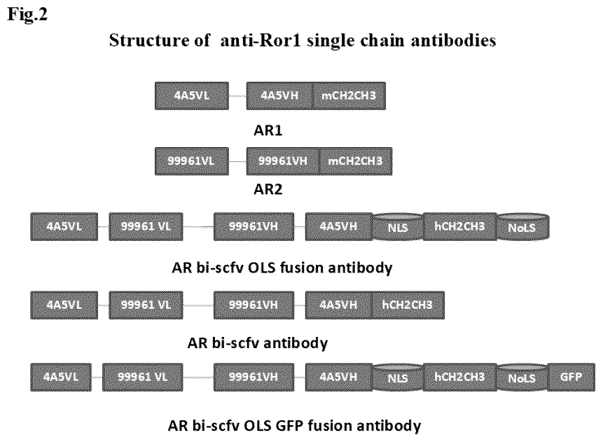

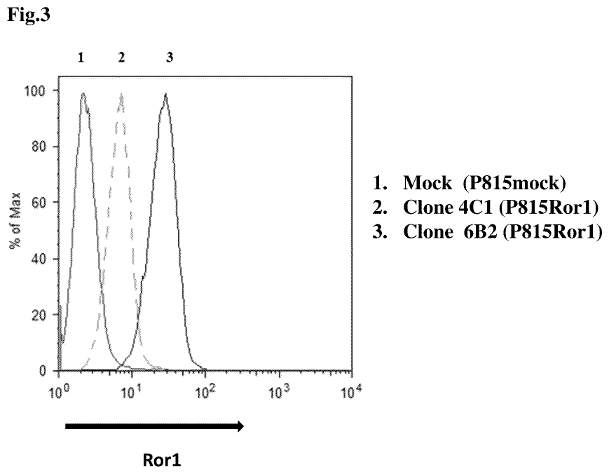

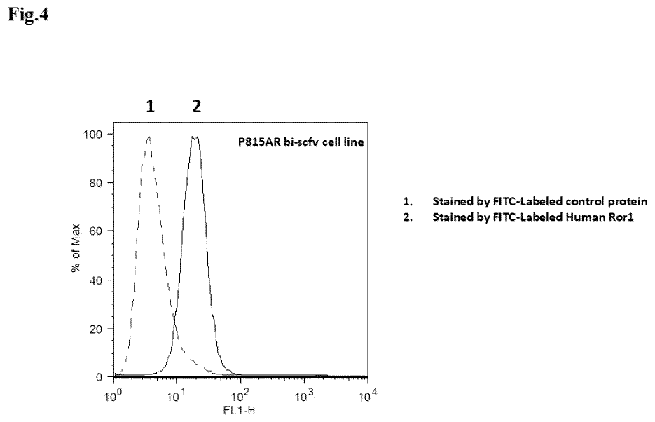

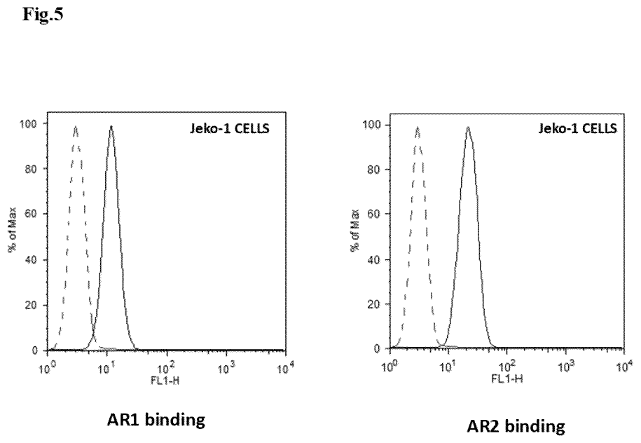

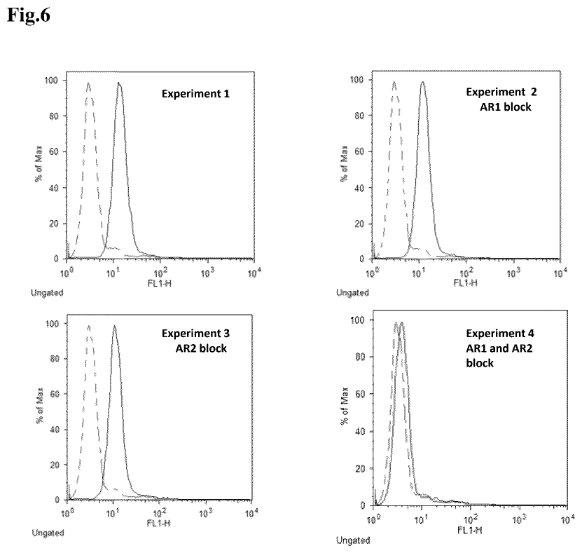

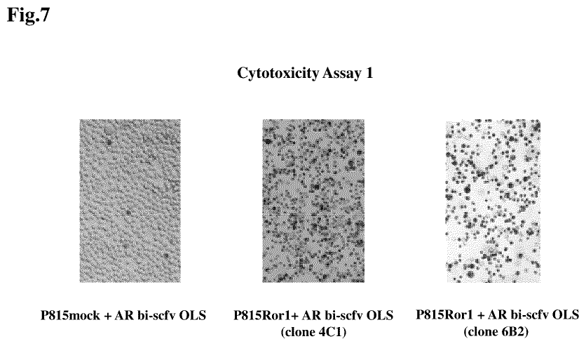

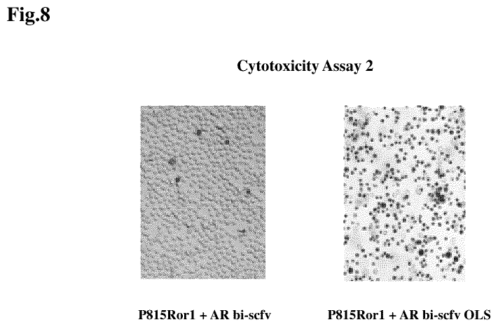

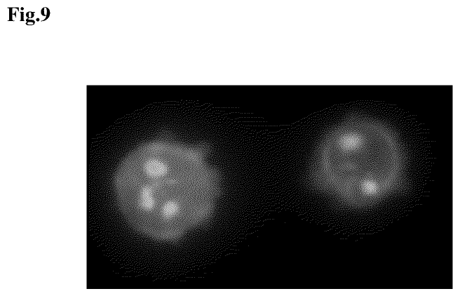

In order to generate an anti-Ror1 biparatopic bi-specific scfv OLS fusion antibody (a practical example of OLS fusion antibody for testing the new cell-killing method), two anti-Ror1 mono-specific scfv antibodies were initially generated. The sequence of first anti-Ror1 mono-specific scfv antibody (named as AR1) was derived from a published anti-Ror1 full length antibody (antibody 4A5, U.S. Patent# US20130273073A1). The sequence of second anti-Ror1 mono-specific scfv antibody (named as AR2) was derived from another published anti-Ror1 full length antibody (antibody 99961, U.S. Patent# US9758591B2). According to published patents, (i) both AR1 and AR2's parental full length antibodies bound to the extracellular domain of Ror1 protein, but at two non-overlap epitopes; (ii) both AR1 and AR2's parental full length antibodies can induce receptor-mediated antibody internalization after binding with Ror1 protein. The structures of AR1 and AR2 were shown in FIG. 2: (i) 4A5VL and 4A5VH were designed as AR1's Ror1-binding domain; (ii) 99961VL and 99961VH were designed as AR2's Ror1-binding domain; (iii) mCH2CH3 fragment in both AR1 and AR2 was designed as a purification and detection tag.

The nucleotides encoding AR1 or AR2 were synthesized by IDT Inc (Integrated DNA Technologies) and sub-cloned to a PCDNA3.1 vector (Invitrogen, CA) according to the general techniques of genetic engineering and molecular cloning detailed in Molecular Cloning, A Laboratory Manual, 3rd edition, Cold Spring Harbor Laboratory Press, Cold Spring Harbor, N.Y. (2001). The sequences of sub-cloned gene fragments (containing nucleotides encoding a signal peptide in the N terminus and a stop code in the C terminus) were verified by DNA sequencing (IDT Inc). The resulting DNA construct was named as PCDNA3-AR1 or PCDNA3-AR2 plasmid. PCDNA3-AR1 and PCDNA3-AR2 plasmids were respectively used to express two different anti-Ror1 mono-specific scfv antibodies, AR1 and AR2.

AR1 and AR2 were produced by CHO cells (Cat#R80007, Invitrogen, Carlsbad, CA) according to manufacturer's protocol. Briefly, the sequence verified plasmids (PCDNA3-AR1 and PCDNA3-AR2) were respectively used to transfect CHO cells. FreeStyle™ MAX Reagent (Cat#16447100, Invitrogen, CA) was used as the transfection reagent. Transfected CHO cells were cultivated in serum-free FreeStyle™ CHO Expression Medium in an incubator at 37° C., 95% humidity and 8% CO2. Transfected CHO cells were cultivated in a cell culture incubator for 7 days on an orbital shaker platform rotating at 135 rpm. AR1 and AR2 were purified from cell culture medium by protein A/G agarose (Cat#20421, Thermo Fisher) according to user manual. The purposes of generating AR1 and AR2 were: (i) before constructing an anti-Ror1 biparatopic bi-specific scfv OLS fusion antibody, two of its parental anti-Ror1 mono-specific scfv antibodies (AR1 and AR2) must first be generated and functionally validated; (ii) mono-specific AR1 and AR2 can be used as the blocking reagents in Flow Cytometry experiments to validate that the anti-Ror1 biparatopic bi-specific scfv OLS fusion antibody, which was derived from AR1 and AR2, can bind to Ror1 protein simultaneously at two non-overlap epitopes.

### 3. Production of Anti-Ror1 Biparatopic Bi-Specific Scfv OLS Fusion Antibody (AR Bi-Scfv OLS Fusion Antibody, an Antibody With OLS Fragment)

It was well known that a cell surface receptor cross-linked by its relevant antibody can induce the cellular internalization of its relevant antibody (J cheng et al, Antibodies 2020, 9(3), 49). Thus, an anti-Ror1 biparatopic bi-specific scfv antibody was designed for the purpose of cross-linking Ror1 protein (a cell surface receptor) and subsequently transporting more antibodies into the target cells through the induction of antibody internalization. In addition, in order to kill the target cells through clogging or over-occupying an essential organelle by the designed anti-Ror1 scfv antibody, an organelle localization sequence (OLS) was added into this anti-Ror1 scfv antibody, named as AR bi-scfv OLS fusion antibody, for the goal of further directing this anti-Ror1 scfv antibody into an essential organelle of target cells. As shown in FIG. 2, the designed AR bi-scfv OLS fusion antibody comprised a cell binding moiety (4A5VL-99961VL-99961VH-4A5VH), an organelle localization sequence (OLS), and a human derived CH2CH3 fragment. The cell binding moiety portion of AR bi-scfv OLS fusion antibody was derived from two mono-specific scfv antibodies, AR1 and AR2. The OLS portion of AR bi-scfv OLS fusion antibody comprised a non-classical NLS and a non-classical NoLS. The non-classical NLS was derived from human HnRNPA1 protein (NP-002127.1, aa 268-aa 305). The non-classical NoLS was derived from human NPM1 protein (NP-002511.1, aa 258-aa 294). The hCH2CH3 fragment in AR bi-scfv OLS fusion antibody was designed as a purification and detection tag.

The nucleotides encoding AR bi-scfv OLS fusion antibody were generated by PCR using PCDNA3-AR1 and PCDNA3-AR2 plasmids as PCR templates. The hCH2CH3 and OLS fragment (including NLS and NoLS) was synthesized by IDT Inc. The synthesized hCH2CH3 and OLS fragment was added to AR bi-scfv portion by overlap PCR. PCR oligos were synthesized by IDT Inc. The resulting PCR product was sub-cloned to a PCDNA3.1 vector. The sequences of sub-cloned PCR fragment were verified by DNA sequencing. The resulting DNA construct was named as PCDNA3-AR bi-scfv OLS plasmid. PCDNA3-AR bi-scfv OLS plasmid was used to express AR bi-scfv OLS fusion antibody.

AR bi-scfv OLS fusion antibody was produced in CHO cells by the same procedure which was used to produce AR1 and AR2. AR bi-scfv OLS fusion antibody was purified from cell culture medium by protein A agarose (Cat#20333, Thermo Fisher) according to user manual. In the process of AR bi-scfv OLS fusion antibody production, it was observed that a large amount of AR bi-scfv OLS fusion antibody was constrained in the nucleus (likely due to the existence of OLS fragment), and that only a small amount of AR bi-scfv OLS fusion antibody was secreted out into the cell culture medium.

### 4. Production of Anti-Ror1 Biparatopic Bi-Specific Scfv Antibody (AR Bi-Scfv Antibody, an Antibody Without OLS Fragment)

An anti-Ror1 biparatopic bi-specific scfv antibody, named as AR bi-scfv antibody, was also generated in order to explore the cell-killing effect by an anti-Ror1 scfv antibody without OLS fragment. As shown in FIG. 2, the designed AR bi-scfv antibody comprised: 4A5VL-99961VL-99961VH-4A5VH-hCH2CH3. The Nucleotides encoding AR bi-scfv antibody were generated by PCR using PCDNA3-AR bi-scfv OLS plasmid as the PCR template. The resulting PCR product was sub-cloned to a PCDNA3.1 vector. The sequences of sub-cloned PCR fragment were verified by DNA sequencing. The resulting DNA construct was named as PCDNA3-AR bi-scfv plasmid. PCDNA3-AR bi-scfv plasmid was used to express AR bi-scfv antibody. AR bi-scfv antibody was produced in CHO cells by the same procedure which was used to produce AR bi-scfv OLS fusion antibody.

### 5. Production of AR Bi-Scfv OLS GFP Fusion Antibody

In order to probe the cell-killing mechanism through detecting the cellular localization of AR bi-scfv OLS fusion antibody, a GFP (green fluorescent protein) fragment was genetically added at the C terminus of AR bi-scfv OLS fusion antibody, named as AR bi-scfv OLS GFP fusion antibody. The structure of AR bi-scfv OLS GFP fusion antibody was shown in FIG. 2. The nucleotides encoding AR bi-scfv OLS GFP fusion antibody were generated by overlap PCR using PCDNA3-AR bi-scfv OLS plasmid and PCDNA3-GFP plasmid as PCR templates. The PCR product was sub-cloned to a PCDNA3.1 vector. The sequences of sub-cloned PCR fragment were verified by DNA sequencing. The resulting DNA construct was named as PCDNA3-AR bi-scfv OLS GFP plasmid. PCDNA3-AR bi-scfv OLS GFP plasmid was used to express AR bi-scfv OLS GFP fusion antibody. AR bi-scfv OLS GFP fusion antibody was produced in CHO cells by the same procedure which was used to produce AR bi-scfv OLS fusion antibody.

### 6. Generation of P815Ror1 Cells

In order to avoid that Ror1's original function (such as inhibiting apoptosis or promoting cell growth) might influence the cell-killing effect exerted by AR bi-scfv OLS fusion antibody, a stable Ror1-expressing cell line, name as P815Ror1, was generated from a mouse derived Ror1 negative cell line (P815, ATCC#TIB-64). P815Ror1 cells were used herein as the target cells for cell-killing assay.

P815Ror1 cells were generated by transducing P815 cells with Ror1-expressing Lenti-virus. A Ror1 plasmid (Cat: HG13968-UT, Sino Biological, NM_005012.3, human Ror1 gene) was used as the template to generate Ror1 PCR product. Ror1 PCR product was inserted into a Lenti-viral vector PLEF1a (Biosettia Inc, CA) according to manufacturer's protocol. The resulting DNA construct was named as PLEF1a-Ror1 plasmid. All plasmids used in Lenti-virus production were purified by QIAGEN Endo-free Maxi prep kit (Cat#12362). Ror1-expressing Lenti-viral particles were packaged by human embryonic kidney cells 293T. Twenty four hours before transfection, 293T cells were seeded at 12×10e6 per T150 tissue culture flask, and transfected with 20 ug pVSVG, 20 ug pRSV REV, 20 ug of pMDLg/p. RRE, and 60 ug of sequenced PLEF1a-Ror1 plasmid in the following day. Lipofectamine 2000 (Cat#11668019, Invitrogen) was used as the transfection reagent. The resulting virus supernatant was harvested at twenty four and forty eight hours post-transfection and combined. Ror1-expressing Lenti-viral particles were concentrated by ultracentrifugation at 28,000 rpm for three hours with a Beckman SW28 rotor (Beckman Coulter). The concentrated Ror1-expressing Lenti-viral particles were used to transduce P815 cells overnight in the presence of 8 μg/ml polybrene. When used for Lenti-viral transduction, P815 cells were cultured in Dulbecco's modified Eagle's medium (DMEM, Cat#11965092 Thermo Fisher) supplemented with 10% fetal bovine serum (FBS), in a 5% CO2 incubator at 37° C. Twenty four hours post-transduction, cells were selected with 2 μg/ml puromycin. Transduced and puromycin-selected cells were stained by BD Pharmingen™ PE Mouse Anti-Human Ror1 mAb (Cat#: 564474) according to user manual and sorted into 96 well plates one cell per well by Flow Cytometry. Sorted and puromycin-selected cells stably expressing Ror1 protein were named as P815Ror1 cells. At the same time, P815 mock cells (Ror1 negative control cells) were also generated by transducing p815 cells with Lenti-viral particles which packaged an empty Lenti-viral PLEF1a vector. The procedure for generating P815 mock cells was the same as that for generating P815Ror1 cells. Before used for further experiments, two single P815Ror1 stable expression clones (clone 4C1 and clone 6B2) with similar growth rate were examined by Flow Cytometry to re-check the expression of Ror1 protein after the process of single clone expansion. 1×10e6 clone 4C1 cells, clone 6B2 cells, or P815 mock cells were stained with BD Pharmingen™ PE Mouse Anti-Human Ror1 mAb, washed three times with FACS buffer, and then processed to Flow Cytometry. Flow Cytometry was performed on a FACS-Calibur apparatus (BD BioSciences, Mountain View, CA). CellQuest software was used to acquire FACS data (BD BioSciences, Mountain View, CA). FACS staining and measuring of the fluorescence intensity were performed as described in Current Protocols in Immunology (Coligan, Kruisbeek, Margulies, Shevach and Stroher, Wiley-Interscience, 2002). All Flow Cytometry data were analyzed with FlowJo software (TreeStar, San Carlos, CA). As shown in FIG. 3, (i) there were clear FACS (Fluorescent Activated Cell Sorting) Histogram shifts between P815 mock cells and P815Ror1 cells, which indicated that P815Ror1 cells from both sorted single clones (clone 4C1 and clone 6B2) had a stable Ror1 protein expression; (ii) there was more FACS Histogram shift in clone 6B2 cells than in clone 4C1 cells, which indicated that clone 6B2 cells expressed a higher level of Ror1 protein than clone 4C1 cells.

### 7. Generation of P815AR Bi-Scfv Cells

AR bi-scfv OLS fusion antibody comprised AR bi-scfv portion (cell binding moiety portion) and OLS portion (organelle localization sequence portion). In order to rule out that the AR bi-scfv portion of AR bi-scfv OLS fusion antibody might be toxic to P815 cells, a stable AR bi-scfv antibody expressing cell line, name as P815 AR bi-scfv, was generated from a mouse derived cell line (P815). The rationale behind this experiment was: if AR bi-scfv antibody can be stably expressed by P815 cells, the AR bi-scfv portion of AR bi-scfv OLS fusion antibody would not be toxic to P815 cells.

The procedure for generating P815 AR bi-scfv cells was the same as that for generating P815Ror1 cells. PCDNA3-AR bi-scfv plasmid was used as the PCR template to generate Lenti-viral AR bi-scfv expressing construct. The Lenti-viral AR bi-scfv PCR product (which included: 4A5VL-99961VL-99961VH-4A5VH-hCH2CH3) was inserted into a Lenti-viral vector PLEF1a. The resulting DNA construct was named as PLEF1a-AR bi-scfv plasmid. PLEF1a-AR bi-scfv plasmid was used to producing AR bi-scfv Lenti-viral particles. AR bi-scfv Lenti-viral particles were used to transduce P815 cells. Twenty four hours post-transduction, cells were selected with 2 μg/ml puromycin. 1×10e6 transduced and puromycin-selected cells were first fully washed with FACS buffer for three times, stained with 100 μL of 10μg/mL of FITC-Labeled Ror1 protein (Cat#RO1-HF253, ACROBiosystems) or negative control protein (Cat#BC7-H5254, ACROBiosystems) according to manufacturer's protocol, washed with FACS buffer for three times, then processed to Flow Cytometry. Flow Cytometry was performed on a FACS-Calibur apparatus. CellQuest software was used to acquire FACS data. Flow Cytometry data were analyzed with FlowJo software. As shown in FIG. 4, there was clear FACS Histogram shift between solid line (cells stained by Ror1 protein) and dash line (cells stained by negative control protein). Flow Cytometry data shown in FIG. 4 demonstrated that AR bi-scfv antibody can be stably expressed by P815 cells, which indicated that the AR bi-scfv portion of AR bi-scfv OLS fusion antibody was not toxic to P815 cells.

The OLS portion of AR bi-scfv OLS fusion antibody contained a NLS and a NoLS. The sequences of NLS and NoLS used herein (respectively derived from human HnRNPA1 and NPM1 proteins) were exactly the same between human and mouse. Thus, the OLS portion of AR bi-scfv OLS fusion antibody cannot be toxic to the mouse derived P815 cells.

Therefore, both AR bi-scfv portion and OLS portion of AR bi-scfv OLS fusion antibody were not toxic to P815 cells.

### 8. Binding Validation of AR1 and AR2

Before combining two anti-Ror1 mono-specific scfv antibodies (AR1 and AR2) into one anti-Ror1 biparatopic bi-specific scfv antibody (AR bi-scfv OLS fusion antibody), AR1 and AR2were first validated for their Ror1-binding function by Flow Cytometry.

Jeko-1 cells (ATCC #CRL-3006 TM) were human derived Ror1 positive cells (Blood 2010 November 25; 116(22); 4532-4541). AR1 and AR2 comprised a mouse derived CH2CH 3 fragment which was used as the purification and detection tag (as shown in FIG. 2). In order to avoid the possible staining interference resulted from goat anti-mouse secondary antibody, in this Flow Cytometry experiment, human derived Ror1 positive Jeko-1 cells (not mouse derived Ror1positive P815 Ror1 cells) were used as the testing cells to validate AR1 and AR2's Ror1-binding function.

AR1 and AR2's parental full length antibodies (antibody 4A5 and antibody 99961) were two published and well characterized anti-Ror1 antibodies (U.S. patents: US20130273073A1 and U.S. Pat. No. 9,758,591B2). Thus, the present invention only used Jeko-1 cells (Ror1 positive cells) as the testing cells in this Flow Cytometry experiment to validate AR1 and AR2's Ror1-binding function. Unlike other Flow Cytometry experiments which used a Ror1 negative cell as the Ror1-binding negative control, in this Flow Cytometry experiment, Jeko-1 cells only stained with secondary antibody were used as the Ror1-binding negative control.

1×10e6 Jeko- 1 cells were stained with 100 ng AR1 or AR2 for 30 minutes, washed with FACS buffer three times, stained with goat anti-mouse IgG (H+L) FITC secondary antibody (R&D systems, cat# F0103B) according to manufacturer's protocol, washed with FACS buffer three times, and then processed to Flow Cytometry. Flow Cytometry was performed on a FACS-Calibur apparatus. CellQuest software was used to acquire FACS data. Flow Cytometry data were analyzed with FlowJo software. As shown in FIG. 5, there were clear FACS Histogram shifts between solid line (cells first stained by AR1 or AR2, then stained by secondary antibody) and dash line (cells only stained by secondary antibody), which indicated that both AR1 and AR2 had clear binding to Ror1 protein, and that both AR1 and AR2 were functional anti-Ror1 scfv antibodies.

### 9. Binding Validation of AR Bi-Scfv OLS Fusion Antibody

AR bi-scfv OLS fusion antibody was constructed from two anti-Ror1 mono-specific scfv antibodies (AR1 and AR2) which respectively targeted two non-overlap epitopes within the extracellular domain of Ror1 protein. In order to validate that AR bi-scfv OLS fusion antibody (a structurally designed anti-Ror1 biparatopic bi-specific scfv antibody) can bind Ror1 protein and also can simultaneously bind Ror1 protein at two non-overlap epitopes, four Ror1-binding tests depicted in FIG. 6 were performed: (i) in experiment 1, P815 Ror1 cells (solid line) or P815 mock cells (dash line) were stained with 100 ng AR bi-scfv OLS fusion antibody, washed with FACS buffer three times, stained with mouse anti-human IgG Fc FITC-conjugated secondary antibody (Cat# 9040-02, Southern Biotech) according to manufacturer's protocol, washed with FACS buffer three times, and then processed to Flow Cytometry; (ii) in experiment 2, P815 Ror1 cells (solid line) or P815 mock cells (dash line) were first incubated with excessive AR1 (10 ug/per reaction) for blocking a Ror1 epitope recognized by AR1, washed with FACS buffer three times, stained with 100 ng AR bi-scfv OLS fusion antibody, washed with FACS buffer three times, stained with mouse anti-human IgG Fc FITC-conjugated secondary antibody, washed with FACS buffer three times, then processed to Flow Cytometry; (iii) in experiment 3, P815 Ror1 cells (solid line) or P815 mock cells (dash line) were first incubated with excessive AR2 (10 ug/per reaction) for blocking another Ror1 epitope recognized by AR2, washed with FACS buffer three times, stained with 100 ng AR bi-scfv OLS fusion antibody, washed with FACS buffer three times, stained with mouse anti-human IgG Fc FITC-conjugated secondary antibody, washed with FACS buffer three times, then processed to Flow Cytometry; (iv) in experiment 4, P815 Ror1 cells (solid line) or P815 mock cells (dash line) were first incubated with excessive AR1 and AR2 (both 10 ug/per reaction) for blocking two Ror1 epitopes respectively recognized by AR1 and AR2, washed with FACS buffer three times, stained with 100 ng AR bi-scfv OLS fusion antibody, washed with FACS buffer three times, stained with mouse anti-human IgG Fc FITC-conjugated secondary antibody, washed with FACS buffer three times, then processed to Flow Cytometry. In order to avoid that AR1, AR2, and AR bi-scfv OLS fusion antibody might be internalized into cells when binding to cell surface protein Ror1, four Flow Cytometry experiments were carefully performed on ice and antibody staining time was kept for 20 minutes. The Flow Cytometry results were shown in FIG. 6: (i) in experiment 1, there was clear FACS Histogram shift between solid line and dash line, which indicated that AR bi-scfv OLS fusion antibody can bind to Ror1 protein; (ii) in experiment 2, there was clear FACS Histogram shift between solid line and dash line, which indicated that although being used in excessive amount (10 ug/per reaction), AR1 on itself cannot block the specific binding between AR bi-scfv OLS fusion antibody and Ror1 protein; (iii) in experiment 3, there was clear FACS Histogram shift between solid line and dash line, which indicated that although being used in excessive amount (10 ug/per reaction), AR2 on itself also cannot block the specific binding between AR bi-scfv OLS fusion antibody and Ror1 protein; (iv) in experiment 4, there was no significant FACS Histogram shift between solid line and dash line, which indicated that a combination of AR1 and AR2 can block the specific binding between AR bi-scfv OLS fusion antibody and Ror1 protein. Data shown in FIG. 6 indicated that AR bi-scfv OLS fusion antibody had two functional Ror1-binding domains which can simultaneously bind to two non-overlap Ror1 epitopes, and that AR bi-scfv OLS fusion antibody was a functional anti-Ror1 biparatopic bi-specific scfv antibody.

### 10. Cell-Killing Assay Using AR Bi-Scfv OLS Fusion Antibody (an Antibody With OLS Fragment)

After its double Ror1-binding function confirmed, AR bi-scfv OLS fusion antibody was used to treat P815 Ror1 cells or P815 mock cells for testing its cell-killing effect.

Because the serum concentration of cell culture medium can influence the sensitivity of cell-killing assay (cells cultured in medium with high serum concentration have a better chance to survive due to the over-supply of growth factors from the medium in high serum concentration), for proof of concept, P815 Ror1 cells and P815 mock cells were cultured in a low serum cell culture medium (2% FBS, not normally 10% FBS) when used in cell-killing assay.

On day one, 2×10e4 P815 Ror1 or P815 mock cells were cultured in T75 tissue culture flask with 15 ml cell culture medium in 2% FBS comprising 200 ng/mL AR bi-scfv OLS fusion antibody, in a 5% CO2 incubator at 37° C. For every two days, AR bi-scfv OLS fusion antibody was replenished into the initial 15 ml cell culture medium (each time adding lug AR bi-scfv OLS fusion antibody diluted in 5 ml fresh 2% FBS cell culture medium). Cells were cultured for 12 days in T75 tissue culture flask without completely replacing the old medium with new fresh medium or splitting cells into new tissue culture flasks, only adding 5 ml fresh 2% FBS cell culture medium comprising AR bi-scfv OLS fusion antibody to the original tissue culture flask for antibody replenishment. On day 12, cell viability was determined by Trypan Blue staining (Cat#15250061, Thermo Fisher) according to manufacturer's protocol. A 0.4% solution of Trypan blue (in phosphate-buffered saline, pH 7.2) was added to cells at 1:1 ratio (Trypan blue solution vs cell culture medium) and then immediately took the cell pictures under a microscope at low magnification. If cells were stained by Trypan blue, they were non-viable. The experimental results were presented in FIG. 7: (i) as shown by all three FIG. 7 pictures, after treated with AR bi-scfv OLS fusion antibody for 12 days, P815 Ror1 cells (both clone 4C1 cells and clone 6B2 cells) were extensively stained by Trypan blue, while P815 mock cells were rarely stained by Trypan blue, which indicated that AR bi-scfv OLS fusion antibody can kill P815 Ror1 cells, but had little killing effect on P815 mock cells over a period of 12 days; (ii) as shown by FIG. 7 middle and right side pictures, after treated with AR bi-scfv OLS fusion antibody for 12 days, the remaining cells from clone 6B2 were far less than the remaining cells from clone 4C1, which indicated that AR bi-scfv OLS fusion antibody had better cell-killing and/or better cell growth inhibition effect on cells expressing a higher level of Ror1 protein (as disclosed in FIG. 3, clone 6B2 cells expressed a higher level of Ror1 protein than clone 4C1 cells). To sum up, the data shown in FIG. 7 demonstrated: (i) after treating cells over a long period of time (12 days), AR bi-scfv OLS fusion antibody can kill Ror1 positive cells, but not Ror1 negative cells; (ii) the cell-killing and/or cell growth inhibition effect of AR bi-scfv OLS fusion antibody was dependent on the existence of Ror1 protein; (iii) the cell-killing and/or cell growth inhibition efficiency of AR bi-scfv OLS fusion antibody was correlated with the expression level of Ror1 protein; (iv) because P815 cells endogenously did not have Ror1 protein, the killing and/or growth inhibition of P815 Ror1 cells (which were genetically engineered to express exogenous Ror1 protein) by AR bi-scfv OLS fusion antibody had nothing to do with Ror1 protein's original function (such as inhibiting apoptosis or promoting cell growth).

### 11. Cell-killing Assay Using AR Bi-Scfv Antibody (an Antibody Without OLS Fragment)

In order to find out whether the anti-Ror1 biparatopic bi-specific scfv antibody itself can kill Ror1 positive cells or not, an AR bi-scfv antibody (an anti-Ror1 biparatopic bi-specific scfv antibody without OLS fragment) was generated and used to treat P815 Ror1 cells. In this experiment, AR bi-scfv OLS fusion antibody (an anti-Ror1 biparatopic bi-specific scfv antibody with OLS fragment) was used as the positive control.

On day one, 2×10e4 P815 Ror1 cells were cultured in T75 tissue culture flask with 15 ml cell culture medium in 2% FBS comprising 200 ng/ml AR bi-scfv antibody or AR bi-scfv OLS fusion antibody, in a 5% CO2 incubator at 37° C. Two T75 tissue culture flasks with P815 Ror1 cells were prepared for this experiment, one added with AR bi-scfv antibody, the other added with AR bi-scfv OLS fusion antibody. For every two days, AR bi-scfv antibody or AR bi-scfv OLS fusion antibody was replenished into the initial 15 ml cell culture medium (each time adding lug corresponding antibody diluted in 5 ml fresh 2% FBS cell culture medium). Cells were cultured for 12 days in T75 tissue culture flask without completely replacing the old medium with new fresh medium or splitting cells into new tissue culture flasks, only adding 5 ml fresh 2% FBS cell culture medium comprising corresponding antibody to the original tissue culture flask for antibody replenishment. On day 12, cell viability was determined by Trypan Blue staining. The experimental results were presented in FIG. 8: after 12 days, P815 Ror1 cells treated with AR bi-scfv OLS fusion antibody were extensively stained by Trypan blue, while P815 Ror1 cells treated with AR bi-scfv antibody (the same biparatopic bi-specific scfv antibody but without OLS fragment) were rarely stained by Trypan blue. These data indicated that AR bi-scfv OLS fusion antibody can kill P815 Ror1 cells, while AR bi-scfv antibody cannot kill P815 Ror1 cells, which proved that the anti-Ror1 biparatopic bi-specific scfv antibody must comprise an OLS fragment to exert its cell-killing effect on P815 Ror1 cells.

### 12. Mechanism of Action by AR Bi-Scfv OLS Fusion Antibody

In order to explore the mechanism of action, an AR bi-scfv OLS GFP fusion antibody was generated by genetically adding a GFP fragment at the C-terminus of AR bi-scfv OLS fusion antibody. AR bi-scfv OLS fusion antibody tagged by GFP can be used to directly observe its cellular localization after binding to cell surface protein Ror1, which can be used to probe the mechanism of action by AR bi-scfv OLS fusion antibody.

On day one, 2×10e4 P815 Ror1 cells were cultured in T75 tissue culture flask with 15 ml cell culture medium in 2% FBS comprising 200 ng/mL AR bi-scfv OLS GFP fusion antibody, in a 5% CO2 incubator at 37° C. For every two days, AR bi-scfv OLS GFP fusion antibody was replenished into the initial 15 ml cell culture medium (each time adding lug AR bi-scfv OLS GFP fusion antibody diluted in 5 ml fresh 2% FBS cell culture medium). Cells were cultured for 12 days in T75 tissue culture flask without completely replacing the old medium with new fresh medium or splitting cells into new tissue culture flasks, only adding 5 ml fresh 2% FBS cell culture medium comprising AR bi-scfv OLS GFP fusion antibody to the original tissue culture flask for antibody replenishment. On day 12, the remaining P815 Ror1 cells were examined by Fluorescence microscope. As shown in FIG. 9, AR bi-scfv OLS GFP fusion antibody was observed both on the cell surface (a typical cell membrane staining observed) and in the nucleolus (a typical nucleolus staining observed).

Based on above experimental data, the mechanism of action by AR bi-scfv OLS fusion antibody on P815 Ror1 cells was explained as: the nucleolus of P815 Ror1 cells was clogged or over-occupied by the continuous accumulation of external non-toxic AR bi-scfv OLS fusion antibody (P815 Ror1 cells were treated continuously for 12 days with AR bi-scfv OLS fusion antibody), which influenced the vital cellular activities occurring in this essential organelle (nucleolus) and ultimately led to cell death and/or cell growth inhibition.

### III. Conclusion

The presented experimental data prove that continuously accumulating an external non-toxic OLS fusion antibody in an essential organelle (such as nucleolus, which has a highly organized structure and is vital to cell survival) can selectively kill the target cells and/or selectively inhibit the growth of target cells which express a cell surface antigen recognized by this OLS fusion antibody. The cell-killing and/or cell growth inhibition effect of OLS fusion antibody is dependent on the existence of a cell surface antigen which is recognized by this OLS fusion antibody. The cell-killing and/or cell growth inhibition efficiency of OLS fusion antibody is correlated with the expression level of a cell surface antigen which is recognized by this OLS fusion antibody. In addition, the cell-killing and/or growth inhibition effect of OLS fusion antibody has nothing to do with the target antigen's original function.

### REFERENCES

Global, regional, and national life expectancy, all-cause mortality, and cause-specific mortality for 249 causes of death, 1980-2015: a systematic analysis for the Global Burden of Disease Study 2015. Lancet (2016); oct 8; 388:1459-14544.
Nina Shah, Ajai Chari, Emma Scott, et al. B-cell maturation antigen (BCMA) in multiple myeloma: rationale for targeting and current therapeutic approaches. Leukemia (2020); volume 34, pages 985-1005.
Nida Iqbal, Naveed Iqbal. Human Epidermal Growth Factor Receptor 2 (HER2) in Cancers: Over-expression and Therapeutic Implications. Mol Biol Int. (2014) Sep 7; 2014:852748.
The Cell: second edition, A Molecular Approach. Geoffrey M Cooper. Sunderland (MA): Sinauer Associates; 2000. ISBN-10: 0-87893-106-6.
Orit Lavi. Redundancy: A Critical Obstacle to Improving Cancer Therapy. Cancer Res. (2015) Mar 1; 75(5): 808-812.
Dennis J. Slamon, M.D., Ph.D., Brian Leyland-Jones, M.D., et al. Use of Chemotherapy plus a Monoclonal Antibody against HER2 for Metastatic Breast Cancer That Overexpresses HER2. N Engl J Med (2001); 344:783-792.
José Baselga, M.D., Ph.D., Javier Cortés, M.D., et al. Pertuzumab plus Trastuzumab plus Docetaxel for Metastatic Breast Cancer. N Engl J Med (2012); 366:109-119.
Kamir J. Hiam-Galvez, Breanna M. Allen & Matthew H. Spitzer. Systemic immunity in cancer. Nature Reviews Cancer (2021); volume 21, pages 345-359.
Karin E de Visser, Johanna A Joyce. The evolving tumor microenvironment: From cancer initiation to metastatic outgrowth. Cancer Cell (2023); Mar 13; 41(3):374-403.
Mikhail Binnewies, Edward W. Roberts, Kelly Kersten, et al. Understanding the tumor immune microenvironment (TIME) for effective therapy. Nature Medicine. (2018); volume 24, pages 541-550.
Ming-Zhu Jin, Wei-Lin Jin. The updated landscape of tumor microenvironment and drug repurposing. Signal Transduct Target Ther (2020) ; Aug 25;5(1):166.
G Majno, I Joris. Apoptosis, oncosis, and necrosis. An overview of cell death. Am J Pathol (1995); Jan; 146(1):3-15.
David P. Bazett-Jones, Graham Dellaire. The Functional Nucleus. (2016); Apr 23:29-49. ISBN: 978-3-319-38880-9.
Nirmala Hariharan, Mark A Sussman. Stressing on the nucleolus in cardiovascular disease. Biochim Biophys Acta. (2014); Jun; 1842(6):798-801.
Juane Lu, Tao Wu, Biao Zhang, et al. Types of nuclear localization signals and mechanisms of protein import into the nucleus. Cell Commun Signal. (2021); 19, 60.
Robert M Martina, Gohar Ter-Avetisyanb, Henry D Hercec, et al. Principles of protein targeting to the nucleolus. Nucleus. (2015); 6(4)314-325.
Angela D Ragin, Rashida A Morgan, Jean Chmielewski. Cellular Import Mediated by Nuclear Localization Signal Peptide Sequences. Chemistry & Biology, (2002) August; vol. 8, 943-948.
Wanting Yu, Yang Zhan, Boxin Xue, et al. Highly efficient cellular uptake of a cell-penetrating peptide (CPP) derived from the capsid protein of porcine circovirus type 2. J Biol Chem. (2018); September 28; 293(39): 15221-15232.
Niloofar Khairkhah, Ali Namvar, Azam Bolhassani. Application of Cell Penetrating Peptides as a Promising Drug Carrier to Combat Viral Infections. Molecular Biotechnology (2023); 65:1387-1402.
Julie Gaston, Nicolas Maestrali, Guilhem Lalle, et al. Intracellular delivery of therapeutic antibodies into specific cells using antibody-peptide fusions. Sci Rep. 2019 Dec. 10; 9(1):18688.
Jun-Fang Zhang, Hua-Long Xiong, Jia-Li Cao, et al. A cell-penetrating whole molecule antibody targeting intracellular HBx suppresses hepatitis B virus via TRIM21-dependent pathway. Theranostics. 2018 Jan. 1; 8(2):549-562.
Andrew M Scott, Jedd D Wolchok, Lloyd J Old. Antibody Therapy of Cancer. Nat Rev Cancer. (2012) Mar; 22; 12(4):278-87.
Federico Riccardi, Michele Dal Bo, Paolo Macor, et al. A comprehensive overview on antibody-drug conjugates: from the conceptualization to cancer therapy. Front Pharmacol. (2023); Sep 18:14:1274088.
Zheng Tian, Ming Liu, Ya Zhang, et al. Bispecific T cell engagers: an emerging therapy for management of hematologic malignancies. J Hematol Oncol (2021) 14:75.
Robert C. Sterner & Rosalie M. Sterner. CAR-T cell therapy: current limitations and potential strategies. Blood Cancer Journal (2021); 11:69.
Norah A Alturki. Review of the Immune Checkpoint Inhibitors in the Context of Cancer Treatment. J Clin Med. (2023); Jun 27; 12 (13):4301.
Caitlin M. Tilsed, Scott A. Fisher, Anna K. et al. Cancer chemotherapy: insights into cellular and tumor micro-environmental mechanisms of action. Front. Oncol. (2022); 28. July.
Liu Y P, Zheng C C, Huang Y N, et al. Molecular mechanisms of chemo-and radiotherapy resistance and the potential implications for cancer treatment. MedComm (2020). 2021; 2(3):315-340.
Ting Wu, Yun Dai. Tumor microenvironment and therapeutic response. Cancer Lett. (2017); Feb 28:387:61-68.
Ga bor Barna1, Rudolf Mihalik, Zsolt Csende et al. ROR1 expression is not a unique marker of CLL. Hematol Oncol 2011; 29: 17-21.
Nicholas Borcherding, David Kusner, et al. ROR1, an embryonic protein with an emerging role in cancer biology. Protein & Cell, Volume 5, Issue 7, July 2014, Pages 496-502.
Jackie Cheng, Meina Liang, Miguel F. et al. Molecular Mechanism of HER2 Rapid Internalization and Redirected Trafficking Induced by Anti-HER2 Biparatopic Antibody. Antibodies (2020), 9(3), 49.
Michael Hudecek, Thomas M. Schmitt, Sivasubramanian Baskar, et al. The B-cell tumor-associated antigen ROR1 can be targeted with T cells modified to express a ROR1-specific chimeric antigen receptor. Blood (2010) 116 (22): 4532-4541.

1. A method for selective cell-killing and/or cell growth inhibition through accumulating an OLS fusion antibody (organelle localization sequence fusion antibody) in an organelle of the target cells at the aim of clogging or over-occupying that organelle, wherein said OLS fusion antibody comprises: (i) a cell binding moiety, which selectively binds to a cell surface antigen of the target cells; and (ii) an organelle localization sequence (OLS), which directs the OLS fusion antibody into an organelle of the target cells.

2. The method of claim 1, (i) wherein said a cell binding moiety is an antibody; (ii) wherein said a cell binding moiety is an antigen-binding fragment; (iii) wherein said a cell binding moiety is a protein scaffold; (iv) wherein said a cell binding moiety is a cytokine; (v) wherein said a cell binding moiety is a ligand for receptor.

3. The method of claim 1, (i) wherein said an OLS is a nuclear localization sequence; (ii) wherein said an OLS is a nucleolar localization sequence; (iii) wherein said an OLS is a combination of nuclear localization sequence and nucleolar localization sequence; (iv) wherein said an OLS is an endoplasmic reticulum localization sequence; (v) wherein said an OLS is a mitochondrion localization sequence; (vi) wherein said an OLS is a lysosome localization sequence; (vii) wherein said an OLS is a Golgi apparatus localization sequence.

4. The method of claim 1, (i) wherein said target cells are cells expressing a specific cell surface antigen; (ii) wherein said target cells are cancer cells or tumor cells; (iii) wherein said target cells are immune cells; (iv) wherein said target cells are cells infected by viruses; (v) wherein said target cells are cells infected by bacteria; (vi) wherein said target cells are cells infected by fungi; (vii) wherein said target cells are cells infected by parasites.

5. A pharmaceutical composition for the treatment of a disease or disorder by using the OLS fusion antibody as an active ingredient, wherein said a disease or disorder is associated with a specific cell surface antigen which is recognized by the OLS fusion antibody, and wherein the goal of said treatment is to selectively kill the target cells and/or to selectively inhibit the growth of target cells, which express that specific cell surface antigen, through selectively accumulating the OLS fusion antibody in an organelle of the target cells at the aim of clogging or over-occupying that organelle.

6. The composition of claim 5, (i) wherein said a disease or disorder is a cancer or tumor; (ii) wherein said a disease or disorder is an autoimmune disease; (iii) wherein said a disease or disorder is an infectious disease.

7. A pharmaceutical composition for the treatment of a disease or disorder by using a polynucleotide (which encodes the OLS fusion antibody) as an active ingredient, wherein said a disease or disorder is associated with a specific cell surface antigen which is recognized by the encoded OLS fusion antibody, and wherein the goal of said treatment is to selectively kill the target cells and/or to selectively inhibit the growth of target cells, which express that specific cell surface antigen, through selectively accumulating the OLS fusion antibody in an organelle of the target cells at the aim of clogging or over-occupying that organelle.

8. The composition of claim 7, (i) wherein said a disease or disorder is a cancer or tumor; (ii) wherein said a disease or disorder is an autoimmune disease; (iii) wherein said a disease or disorder is an infectious disease.
